(19) United States
(12) Patent Application Publication
(10) Pub. No.: US 2004/0208926 A1
(43) Pub. Date: Oct. 21, 2004

(54) Solubilized sertraline compositions

(75) Inventors: Dwayne Thomas Friesen, Bend, OR (US); Scott Max Herbig, East Lyme, CT (US); Ravi Mysore Shanker, Groton, CT (US); James Blair West, Bend, OR (US)

Correspondence Address: PFIZER INC., PATENT DEPARTMENT, MS8260-1611, EASTERN POINT ROAD, GROTON, CT, 06340, US

(73) Assignee: Pfizer Inc

(21) Appl. No.: 10/838,929

(22) Filed: May 4, 2004

Related U.S. Application Data
(63) Continuation of application No. 09/380,825, filed on Sep. 7, 1999, now abandoned.
Application No. 09/380,825 is a 371 of international application No. PCT/IB98/00933, filed on Jun. 15, 1998.
(60) Provisional application No. 60/051,413, filed on Jul. 1, 1997.

(51) Int. Cl. A61K 31/137; A61K 9/20

(52) U.S. Cl. 424/465; 514/649

(57) ABSTRACT

Compositions of matter comprising sertraline and a solubilizing agent which increases the solubility of sertraline in aqueous chloride ion-containing use environments.

### FIELD OF THE INVENTION

This invention relates to a composition comprising sertraline or a pharmaceutically acceptable salt thereof and a solubilizing agent which prevents gel formation or otherwise maintains the solubility of sertraline in a use environment containing chloride ions. The invention further relates to a method of treating a psychiatric or other illness comprising administering sertraline in such a solubilized composition to a mammal, including a human patient, in need of such treatment.

### BACKGROUND OF THE INVENTION

Sertraline is a selective serotonin reuptake inhibitor (SSRI), which is useful as an antidepressant and anorectic agent, and in the treatment of obsessive-compulsive disorder, premenstrual dysphoric disorder, post-traumatic stress disorder, chemical dependencies, anxiety-related disorders, panic and premature ejaculation.

Sertraline is most commonly prescribed for therapy of depressive illness, in the general dose range 50-200 mg/day. Sertraline has an elimination half-life of 0.23 hr and is dosed once daily. Commercially, sertraline is available as the hydrochloride salt which is undeniably therapeutically effective, many patients having availed themselves of the benefits of this drug.

Some forms of sertraline, particularly salts which exhibit high solubility, can be problematic, however. Such salts, generally those having an aqueous solubility in excess of 10 mg/mL, can exhibit a tendency to form a gel and/or exhibit reduced solubility (e.g., precipitate as a salt or as the free base having a lower solubility in the environment of use than the salt form originally administered) when exposed to a use environment containing chloride ions such as the gastrointestinal tract. The gel itself tends to dissolve slowly and otherwise releases sertraline at a slow rate, thereby affecting absorption. It is not known whether gelation is the only mechanism which impacts the solubility of sertraline in a use environment. However, therapeutic difficulties can accordingly arise from administering an immediate-release dosage form in vivo if solubility is affected, regardless of mechanism. Problems can similarly arise in the case of controlled-release dosage forms since the controlled release profile of the dosage form can be altered in vivo by factors affecting solubility. The unanticipated phenomenon of gelation of sertraline salts in a chloride ion-containing environment can thus create therapeutic difficulties by unexpectedly altering the release profile of a dosage form, whether immediate-release or controlled-release. The mechanism of sertraline gelation is not well understood, and can be all the more problematic therapeutically since the release characteristics of a gel formed in situ may not be anticipated.

In particular, gelling of sertraline in sustained-release dosage forms can be detrimental in those sustained release systems known as non-eroding matrix systems, reservoir systems, and osmotic systems. In each of these types of sustained release formulations release of the drug is dependent on transport of the drug across a distance within the device (matrix or coating layer) to the surrounding fluid. This drug transport can occur by diffusive or convective mechanisms. In both mechanisms, formation of a gel can reduce transport by an order of magnitude or more and in some cases can result in devices that exhibit incomplete drug release (e.g., less than 70% of the total drug in the formulation).

### SUMMARY OF THE INVENTION

This invention provides a composition of matter, suitable for administration to a mammal, including a human, comprising sertraline or a pharmaceutically acceptable salt thereof and an amount of an excipient, herein termed a “solubilizing agent” sufficient to effect a concentration of dissolved sertraline in a use environment containing chloride ions which is at least 1.5 times higher, preferably 2 times higher, more preferably 3 times higher than the concentration effected by a comparative composition of matter (i.e., a control) identical thereto but for the inclusion of said solubilizing agent. The use environments mainly intended are the aqueous in vivo digestive fluids of the gastrointestinal (GI) tract including the stomach, small intestine and large intestine, and aqueous in vitro chloride ion-containing test media, as further described below. The compositions are suitable for formulating into oral dosage forms including tablets, capsules, multiparticulates, powders for oral suspension, and unit dose packets (sometimes referred to in the art as a “sachet”). In addition the compositions can be used in liquid dosage forms such as oral solutions or suspensions and injectable formulations. For making the compositions of this invention into oral dosage forms, conventional techniques known to the art can be employed. The composition can additionally comprise other conventional pharmaceutical ingredients and/or a pharmaceutically acceptable carrier.

By this invention, it has been determined that in cases of dosage forms containing sertraline salts which form gels or which otherwise exhibit reduced solubility in a use environment, solubility may advantageously be increased, and in some cases solution viscosity may be advantageously decreased, by employing the sertraline salt together with a solubilizing agent which increases the sertraline's solubility. The solubilizing agent preferably also maintains solubility, meaning that the level of dissolved sertraline in a use environment, regardless of the salt employed, is held at a concentration greater than or equal to 1.5 times the concentration of sertraline in a like formulation without solubilizing excipient, for at least 2 hours. For many dosage forms it may be advantageous to maintain the sertraline concentration greater than or equal to 1.5 times the concentration of sertraline in like formulations without solubilizing excipient for longer periods of time such as 4 hours, 8 hours, 16 hours, or 20 hours, and this can be effected by the choice and amount of solubilizing agent. It has otherwise been determined that in a chloride ion-containing use environment without a solubilizing agent, for example a test environment such as 0.075M sodium chloride solution, sertraline solubility is generally less than 10 mgA/mL, usually less than 5 mgA/mL, regardless of the salt employed, and despite the fact that many of the salts themselves exhibit solubilities in pure water (i.e., no chloride ions) well in excess of 10 mgA/mL. Solubilizing agents thus could also be construed to be compounds that maintain sertraline concentrations of 10 mgA/ml or greater in chloride-ion-containing environments of use.

Reference herein to “a solubilizing agent” herein, including the claims, shall be understood as also including the use of more than one solubilizing agent in a composition, added separately or as a mixture.

As mentioned above, the term “use environment” can refer to the aqueous in vivo chloride ion-containing digestive fluids of the stomach, or to an in vitro chloride ion-containing aqueous environment used to test a dosage form for its sertraline release characteristics. A useful in vitro test environment for purposes of this invention is 0.075M sodium chloride. 0.075M sodium chloride is preferred as a test medium because of its ready availability and similar chloride ion concentration to the lower levels of chloride ions found in the fluids in the GI tract. Blood & Other Body Fluids, Dorothy S. Dittmer, ed., Federation of American Societies for Experimental Biology, Washington, D.C., 1961, pp. 404419. Thus, as an additional feature, this invention provides an in vitro test to determine whether a dosage form is within the scope of the invention. That is, the invention provides a composition of matter comprising sertraline or a pharmaceutically acceptable salt thereof and an amount of a solubilizing agent sufficient to produce and to maintain, for at least 2 hours in 0.075M sodium chloride, a concentration of dissolved sertraline which is at least 1.5 times higher than the concentration effected by a comparative composition of matter 0.10 identical thereto but for the inclusion of said solubilizing agent. Agitation should be employed during the test although, as explained below, the degree or type of agitation is not critical. Salt solution temperature is not believed to be particularly critical so long as it is about 37° C., plus or minus 3° C., throughout the test. Excipients, including the solubilizing agent(s) should be at the desired concentration in the aqueous test solution prior to adding sertraline and sodium chloride. Sertraline is then added to a concentration ranging between 80% to 100% of its saturation concentration in the test solution. This solution should be decanted off or filtered away from any solids. To this solution a 3M NaCl solution is slowly added with stirring until the NaCl concentration in the test solution is 0.075M. The sertraline concentration in this test solution after 2 hours is compared with a control solution made in the same manner and consisting of the same components except the solubilizing agent.

Alternatively, a solubilizing excipient can be identified in an in vivo test such as a crossover study. In an in vivo crossover study a solubilized sertraline-containing dosage form is dosed to half a group of 12 or more humans and, after an appropriate washout period (e.g., one week) the same subjects are dosed with a dosage form otherwise identical but for inclusion of the solubilizing agent. The other half of the group is dosed with the non-solubilized dosage form first, followed by the solubilized dosage form. Maximum concentration in the blood (Cₘₐₓ) and/or bioavailability, measured as the area under the curve (AUC) for a plot of the concentration of sertraline in blood versus time, is determined for each group. By comparison, assessment of the solubilized dosage form can be made. If the average Cₘₐₓ or AUC for the formulation containing the solubilizing agent is greater by 10% or more than the formulation without the solubilizing agent, then the solubilizing excipient is an embodiment of this invention. It is preferred that the Cₘₐₓ and/or AUC be greater by at least 15%, and more preferred either or both be greater by at least 20%. The determination of AUC's is a well known procedure and is described, for example, in “Pharmacokinetics; Processes and Mathematics,” by Peter Welling (ACS Monograph 185, Amer. Chem. Soc., Wash. D.C., 1986). Thus, as an additional feature of the invention, the invention provides a composition of matter comprising sertraline or a pharmaceutically acceptable salt thereof and an amount of a solubilizing agent sufficient to effect, in vivo, a Cₘₐₓ and/or an AUC which is greater by at least 10% than the Cₘₐₓ and/or AUC effected by a comarison composition of matter (i.e., a control) identical thereto but for the inclusion of said solubilizing agent.

The invention further provides a method of increasing the solubility of sertraline in an aqueous chloride ion-containing environment, comprising administering said sertraline in a composition of matter comprising sertraline and a solubilizing agent.

The invention is surprising in that, prior to the invention, it was not known that (1) the phenomenon of reduced sertraline solubility in chloride ion-containing environments existed, nor that (2) any chemical agent existed which would reduce or prevent sertraline gelation or reduced sertraline solubility in chloride ion-containing use environments or otherwise operate to increase sertraline's solubility in such use environments. The term “solubilized sertraline” is used herein to refer to a composition comprising sertraline or a sertraline salt plus an excipient (i.e. the solubilizing agent) which prevents gelation or otherwise increases, and preferably maintains, the solubility of the sertraline salt in an in vivo or in vitro chloride ion-containing use environment. Likewise, the term “solubilize” is used to denote that the solubility of a sertraline salt is being increased by at least 1.5 times in a use environment over what it would be in the absence of a solubilizing agent.

The invention is preferred for use with the aspartate, acetate, and lactate salts which are salts that exhibit high solubilities in water relative to the free base. These salts are disclosed in commonly assigned co-pending provisional applications PC9297RTR, PC9298RTR, and PC9856RTR, all filed on the same date as this application, and each of which is herein incorporated by reference.

For convenience and consistency, reference to “sertraline” in terms of therapeutic amounts herein, including the claims, is to active sertraline, abbreviated herein as “mgA”, i.e., the non-salt, non-hydrated free base having a molecular weight of 306.2. Amounts in mgA can conveniently be converted to equivalent weights for whatever salt form is desired.

Many solubilizing agents useful herein can be grouped into several broad categories:

1. Organic acids and organic acid salts;

2. Partial Glycerides, i.e., less than fully esterified derivatives of glycerin, including monoglycerides and diglycerides;

3. Glycerides;

4. Glyceride derivatives;

5. Polyethylene glycol esters;

6. Polypropylene glycol esters;

7. Polyhydric alcohol esters;

8. Polyoxyethylene ethers;

9. Sorbitan esters; and

10. Polyoxyethylene sorbitan esters.

11. Carbonate salts

### DETAILED DESCRIPTION

The amount of solubilizing agent employed in a composition according to the invention depends on the particular solubilizing agent employed.

In the case of solubilizing agents which are organic acids the preferred amount of solubilizer can be calculated as a ratio multiplied by the quantity of sertraline to be used, wherein the ratio is of organic acid solubility to solubility of sertraline salt:

(organic acid or salt solubility/sertraline or sertraline salt solubility)×quantity of sertraline

where the solubilities referred to are in mg/ml. The above expression is approximate, and some adjustment may be advantageous for optimization. Generally the above expression will give a quantity which is plus or minus 25% of the final value employed, although higher quantities of solubilizing agent can be incorporated without any particular additional advantage. In addition, organic acid salts can be added to modify the pH and/or solubility of the organic acid, effectively optimizing the solubilization effect of the agents.

For other types of solubilizing agents listed, typically the amount of solubilizing agent employed in the dosage form will be 1 to 150% by weight of the amount of sertraline employed therein, preferably 1 to 100%, more preferably 3 to 75%. Amounts of solubilizing agent higher than 150% may be employed, although it is believed that in most cases no particular advantage would be provided.

Salts of sertraline or excipients that in combination with sertraline aid in solubilizing sertraline can be beneficial to virtually any type of sertraline dosage forms intended for oral administration, including immediate release as well as controlled release systems, including (1) sustained-release dosage forms which meter out sertraline as they progress through the gastrointestinal system and (2) delayed release systems which release sertraline after an initial delay period following ingestion. Immediate-release systems are well known and commercially available in both solid and liquid formulations. Controlled release dosage forms of sertraline are discussed and disclosed in commonly assigned co-pending provisional applications Pfizer Docket PC9337JTJ and PC9824JTJ, each filed of even date herewith and each herein incorporated by reference in its entirety. Solubilized sertraline can enhance release from the dosage form by increasing the concentration gradient for diffusive based systems such as matrix dosage forms and reservoir dosage forms. Solubilized sertraline can also enhance delivery from osmotic dosage forms in that a more soluble sertraline can increase the osmotic pressure in the core and increase the sertraline concentration in the fluid that is pumped or extruded out of the dosage form. In addition, solubilized sertraline can benefit sustained-release formulations by aiding absorption of drug from the G.I. tract. For example, higher concentrations of drug in the colon can increase absorption due to a higher concentration gradient across the intestinal wall.

It is noted that currently available commercial dosage forms of sertraline are immediate-release dosage forms containing sertraline hydrochloride. Even though the hydrochloride has proven to be very effective, it is possible that dosage forms containing the hydrochloride can also benefit by the addition of a solubilizing agent.

Examples of organic acids useful in the invention include malic, citric, erythorbic, adipic, glutamic, aspartic, maleic, aconitic, and ascorbic acid. Preferred acids are citric, erythorbic, ascorbic, glutamic, and aspartic. Salts of organic acids such as alkalkine earth metal (magnesium, calcium) salts and alkali metal (lithium, potassium, sodium) salts are also effective as well as mixtures of organic acids and their salts. Calcium salts such as calcium carbonate, calcium acetate, calcium ascorbate, calcium citrate, calcium gluconate monohydrate, calcium lactobionate, calcium gluceptate, calcium levulinate, calcium pantothenate, calcium proprionate, calcium phosphate dibasic, and calcium saccharate are preferred organic acid salts.

Examples of compounds within the other categories mentioned above are summarized in Table 1.

TABLE 1
Solubilizing Agents

| Class | Examples, Chemical Name | Examples, Trade Designation, (Vendor) |
|---|---|---|
| Partial | Glyceryl Monocaprylate | Monocaprylin ® (Sigma), Capmul ® |
| Glycerides | | MCM(Abitec), Imwitor ® 308 (Huls) |
| | C8-C10 Partial Glycerides | Capmul ® MCM (Abitec), Imwitor ® 742 |
| | | (Huls), |
| | | Imwitor ® 988 (Huls) |
| | Glyceryl Monooleate | Myverol ® 18-99 (Eastman), Calgene ® |
| | | GMO |
| | | (Calgene), Capmul ® GMO(Abitec) |
| | Glyceryl Monolinoleate | Myverol ® 18-92 (Eastman) |
| | Glyceryl Monostearate | Imwitor ® 191 (Huls) Calgene ® |
| | | GSO(Calgene) |
| | Glycery Monolaurate | Imwitor ® 312 (Huls) Calgene ® GLO |
| | | (Calgene) |
| | Glyceryl Dilaurate | Capmul ® GDL (Abitec) |
| Glycerides | Triacetin | Triacetin (Sigma) |
| Glyceride | PEG-Derivitized Glycerides | Cremophor ® RH40, Cremophor ® RH60 |
| Derivatives | | (BASF), |
| | | Acconon CA5, CA-9, CA-15, W230, |
| | | TGH (Abitec) |
| | Polyglycolized Glycerides | Gelucire ® 44/14, 42/12, 50/13, 53/10, |
| | | 35/10, 48/09, 46/07, 62/05, 50/02; |
| | | Labrasol ® (Gattefosse); Capmul ® 3GO; |
| | | 3GS, 6G2O, 6G2S, 10G4O, 10G10O |
| | | (Abitec) |
| Polyethylene | PEG 200 Monolaurate, | Calgene ® 20-L, Calgene ® 40-L, |
| glycol Esters | PEG 400 Monolaurate, | Calgene ® 60-L |
| | PEG 600 Monolaurate | |
| | PEG 200 Monostearate, | Calgene ® 20-S, Calgene ® 40-S, |
| | PEG 400 Monostearate, | Calgene ® 60-S |
| | PEG 600 Monostearate | |
| | PEG 200 Dilaurate, PEG | Calgene ® 22-L, Calgene ® 42-L |
| | 400 Dilaurate, PEG 600 | Calgene ® 62-L |
| | Dilaurate | |
| Polypropylene | Propylene Glycol | Captex ® 200 (Abitec) |
| Glycol Esters | Dicaprylate | |
| Polyhydric | Diethylene Glycol | Calgene ® DGL |
| Alcohol Esters | Monolaurate | |
| | Propylene Glycol | Calgene ® PGML |
| | Monolaurate | |
| | Ascorbyl Palmitate | Ascorbyl Palmitate (Sigma) |
| Polyoxyethylene | PEG Lauryl Ether | Nonionic L-4 (Calgene) |
| Ethers | | |
| | PEG Stearyl Ether | Nonionic S-20 (Calgene), Myrj 45, 52, |
| | | 53, 59 (Sigma) |
| Sorbitan Esters | Sorbitan Monolaurate | Calgene ® SML, Span ® 20 (Sigma) |
| | Sorbitan Monooleate | Calgene ® SMO, Span ® 80 (Sigma) |
| Polyoxyethylene | POE-20 Sorbitan | Calgene ® PSML-20, Span ® 20(Sigma), |
| Sorbitan | Monolaurate | Tween 20 (Sigma), Capmul ® POE-L |
| Esters | | (Abitec) |
| | POE-20 Monooleate | Tween ® 80, PSMO-20 |
| Saccharide | Sucrose Monolaurate | Ryoto LW-1540 (Chem Service) |
| Esters | | |
| Phospholipids | Phosphatidyl choline | Lecithin (Sigma) |
| | Mixed phospholipids | Emphos D70-30C (Witco) |
| Block Copolymers | PEO-PPO Block | Pluronic ® F-68, F127, L-62 (BASF) |
| | Copolymers | |
| Polyethylene | PEG 3350 | Various sources |
| Glycols | | |

In addition other compounds useful as solubilizing agents in the invention are ethyl propionate, methyl paraben, propyl paraben, propyl gallate, niacinamide, ethyl vanillin, paraaminobenzoic acid, butylated hydroxyanisole, imidurea, and glycine. It is also noted that preferred compositions include mixtures of an organic acid with or without a corresponding organic acid salt, and one or more of the non-organic solubilizers listed above or in Table 1. It is also noted that it has generally been observed that in order to be most effective the solubilizer should have a solubility in the aqueous chloride-ion containing use environment of at least 1 mg/ml, and preferably greater than 5 mg/ml.

A preferred group of solubilizing agents, in addition to the preferred organic acids previously mentioned, includes those in Table 2.

TABLE 2
Preferred Solubilizing Agents

| | Examples, Chemical | |
|---|---|---|
| Class | Name | Examples, Trade Names (source) |
| Partial | Glyceryl monocaprylate | Monocaprylin ® (sigma), Capmul ® |
| Glycerides | | MCM(Abitec), |
| | | Imwitor ® 308 (Huls) |
| | C8-C10 Partial | Capmul ® MCM (Abitec), Imwitor ® 742 |
| | Glycerides | (Huls), |
| | | Imwitor ® 988 (Huls) |
| | Glyceryl Monostearate | Imwitor ® 191 (Huls) Calgene ® |
| | | GSO(Calgene) |
| | Glyceryl Monolaurate | Imwitor ® 312 (Huls) Calgene ® GLO |
| | | (Calgene) |
| Glycerides | Triacetin | Triacetin ® (Sigma) |
| Sorbitan Esters | Sorbitan Monolaurate | Calgene ® SML, Span ® 20 (Sigma) |
| | Sorbitan Monooleate | Calgene ® SMO, Span ® 80 (Sigma) |
| Phospholipids | Phosphatidyl choline | Lecithin ® (Sigma) |
| | Mixed phospholipids | Emphos D70-30C (Witco) |
| Block Copolymers | PEO-PPO Block | Pluronic ® F-68, F127, L-62 (BASF) |
| | Copolymers | |
| Polyethylene | PEG 3350 | Various sources |
| Glycols | | |

Note:
Commercial vendors shown above are as follows:
Abitec Corp. Janesville, WI
BASF, Parsippany, NJ
Calgene Chemical Inc. Skokie, IL
Chem Service, Inc., West Chester, PA
Huls America, Piscataway, NJ
Sigma, St. Louis, MO
Witco, Houston, TX

Preferred combinations of solubilizing agents include (1) an organic acid plus a salt of the same or a different organic acid, (2) an organic acid plus a non-ionic solubilizing agent such as any of those listed in Table 1, and (3) an organic acid plus a salt of the same or a different organic acid plus a non-ionic solubilizing agent.

Particularly preferred individual solubilizing agents include aspartic acid, glyceryl monocaprylate, glyceryl monolaurate, calcium acetate, ascorbic acid, citric acid, glutamic acid, and calcium carbonate. Aspartic acid, glyceryl monocaprylate, glyceryl monolaurate and calcium acetate are most preferred.

As previously discussed, a dosage form can be tested in vitro to determine whether an excipient has a solubilizing effect on sertraline in a chloride-ion containing use environment and thus is useful as a solubilizing agent. A 0.075M NaCl solution is preferred for use as a test medium although other chloride-ion containing solutions with equivalent or higher chloride ion concentration than 0.075M (e.g., 0.1 N HCl or isotonic saline) may be used to determine the solubilizing effect of a test excipient. In some cases reduced solubility is evident simply by adding a dosage form such as a powder to the test medium because gelation is visible. Similar problems may be evident in a dosage form such as a tablet if the tablet is, for example, cut open and gelation is visible on its open face. A recommended procedure is to initially make a solution containing the desired excipients, including solubilizing agent(s). The excipients can be at any concentration relevant to the intended dosage form, but are typically for organic acids and soluble salts or sugars 80-100% of saturation. For other surfactant-like compounds, concentrations typically range from 1 to 150% of the sertraline concentration in the test solution. Sertraline is added to this excipient-containing solution at a concentration typically 80-100% of saturation. The solution is filtered or decanted to remove any solids and then a 3M solution of sodium chloride is added until the sodium chloride concentration is 0.075M. The concentrated sodium chloride solution should be added dropwise with stirring. This test medium should be kept at a temperature on the order of 37° C. for at least 2 hours at which time the sertraline concentration in solution is determined. It is preferred that the sertraline concentration be maintained for 4 hours, more preferably for 8 hours, still more preferably for 16 hours, and most preferably for at least 20 hours. The amount of agitation is not critical. When sampling the test medium, filtration or centrifugation can be employed to obtain solution that is free of any solids or gel material, and also to avoid inclusion of particulates (which may contain sertraline) in the sample. Analysis of the samples to determine sertraline concentration can be accomplished via several conventional analytical methods, such as by high performance liquid chromatography (HPLC). For example, sertraline concentrations can be determined using reverse phase HPLC with a ULTRACARB® 5 ODS 4.6×250 mm column (Phenomonex, Torrance, Calif.), and a mixture of acetic acid, triethylamine, acetonitrile, and water as mobile phase, with UV detection at 230 nm. For example, the mobile phase can be prepared by combining, with stirring, 2.86 ml of glacial acetic acid, 3.48 ml of triethylamine, diluting to a liter with water, and filtering and degassing. Flow rates are typically on the order of 1.5 ml/min, and retention times about 4 minutes.

Dosage forms with solubilizing agent can be formulated by conventional techniques. Immediate release dosage forms can be capsules, tablets, multiparticulates, liquid solutions or suspensions. Capsule formulations can be either soft gelatin capsules where the sertraline is either dissolved or suspended within the capsule core or hard gelatin capsules filled with multiparticulates, tablets or a liquid (solution or suspension) fill. Immediate release tablets can be by techniques standard in the industry by simply including the solubilizing agent as one or more of the tablet excipients. Likewise immediate-release multiparticulates can be made that include solubilizing agents by techniques such as extension spheronization, rotary granulation, coating seed cores or other methods common in the pharmaceutical industry. Liquid formulations consisting of a solution or suspension or both can be made by methods common in the pharmaceutical industry.

Controlled-release dosage forms can also be made that include solubilizing agents by methods common in the pharmaceutical industry. Controlled release dosage forms include a wide variety of dosage forms that impart control over the dissolution rate or rate of release of sertraline from the dosage form. Such dosage forms include but are not limited to sustained release, delayed and then immediate release, delayed and then sustained release and a dosage form with a small portion of sertraline released immediately and then followed by the majority of the sertraline in the dosage release at a sustained rate. Other algorithms of release can also be attained such as pulsitile release. Many such formulations are described in aforementioned co-pending applications PC9337JTJ and PC9824JTJ.

Standard techniques can be used to make controlled release dosage forms. For example, tablets can be made by commonly used direct compression methods that contain sertraline and a solubilizing agent. To provide delayed release, a pH-sensitive coating can be applied to these tablets via a side-vented pan coater (e.g., HCT-60 tablet coater, Vector Corp.). The pH sensitive coating is resistant to low pH environments such as typically in the stomach and then dissolves, releasing sertraline, in neutral pH environment such as typically in the small intestine. Such coating materials (e.g., cellulose acetate phthalate or methacrylic acid copolymer) are common in the pharmaceutical industry. Alternatively, the tablets can be coated with a porous or semipermeable membrane coating to provide sustained release of the tablet cores. A particularly useful process for applying a membrane coating comprises dissolving the coating polymer in a mixture of solvents chosen such that as the coating dries, a phase inversion takes place in the applied coating solution, resulting in a membrane with a porous structure. Numerous examples of this type of coating system are given in European Patent Specification 0 357 369 B1, published Mar. 7, 1990, herein incorporated by reference. Many other types of controlled release dosage forms can also be made that benefit from the inclusion of solubilizing agents such as matrix systems which include but are not limited to 1) non-eroding matrices, tablets, multiparticulates and hydrogel-based systems; 0.2) hydrophilic eroding, dispersible or dissolvable matrix systems, tablets and multiparticulates; and 3) coated matrix systems. Another class of controlled-release dosage forms consists of reservoir systems where release of the drug is modulated by a membrane, such as capsules and coated tablets or multiparticulates. A third class consists of osmotic-based systems such as 1) coated bilayer tablets; 2) coated homogeneous tablet cores; 3) coated multiparticulates; and 4) osmotic capsules. A fourth class consists of swellable systems where drug is release by a swelling and then extrusion of the core components out through a passageway in a coating or surrounding shell or outer layer.

The invention is further illustrated by the following examples, which are not to be taken as limiting.

### EXAMPLE 1

This example illustrates that organic acids have the ability to raise the solubility of the hydrochloride salt of sertraline. The acids were tested by dissolving the candidate acid in water and then stirring excess sertraline hydrochloride in the acid solution for at least 8 hours. The concentration of sertraline in the supernatant was then measured by HPLC analysis. The results of this test are shown in Table 1-1, below. Most of the acids listed in the table successfully raised the solubility of sertraline hydrochloride (normal solubility 2.5 mg/ml).

TABLE 1-1

| | Approximate Excipient | |
|---|---|---|
| Excipient | Concentration (mg/ml) | Sertraline Solubility (mg/ml) |
| D,L-malic acid | 900 | 21 |
| Citric acid | 600 | 20 |
| Erythorbic acid | 400 | 19 |
| Adipic acid | 14 | 12 |
| Maleic acid | 700 | 6.4 |
| L-aspartic acid | 10 | 5.5 |
| Tartaric acid | 1400 | 5.5 |
| L-glutamic acid | 12 | 5.4 |
| Fumaric acid | 11 | 3.1 |
| Tannic acid | 2000 | 2.8 |
| D,L-tyrosine | 600 | 2.2 |

Preferred acids, based on the above-described test, are malic, citric, erythorbic, and adipic acids. Maleic, L-aspartic, tartaric, and L-glutamic acids also significantly improved sertraline hydrochloride solubility. Some controlled-release dosage forms with such acids in the core will perform better than those without such acids. This is particularily true for osmotic-based formulations that deliver a solution of drug.

### EXAMPLE 2

This example illustrates that organic acids have the ability to raise the solubility of the acetate salt of sertraline by a test method similar to that used for the hydrochloride salt described in Example 1. The solubilizing agent, its concentration, and resulting sertraline solubility are shown in Table 2-1 below. Based on these results, preferred acids to include in a dosage form where increased sertraline acetate solubility is desired are ascorbic, erythorbic, citric, lactic, aspartic, glutamic, and aconitic acids.

TABLE 2-1

| | Excipient Concentration | Sertraline Solubility |
|---|---|---|
| Excipient | (mg/ml) | (mg/ml) |
| Ascorbic acid | 400 | >425 |
| Erythorbic acid | 400 | >330 |
| Citric acid | 600 | 146 |
| Lactic acid | 213 | >294 |
| Aspartic acid | 7 | 110 |
| Glutamic acid | 12 | 108 |
| Aconitic acid | 500 | >92 |
| Itaconic acid | 150 | 72 |
| Succinic acid | 77 | 28 |
| None | — | 64 |

### EXAMPLE 3

This example illustrates that organic acids and three calcium salts have the ability to raise the aqueous solubility of the lactate salt of sertraline using a method similar to that used for the hydrochloride salt described in Example 1. The solubilizing agent, its concentration in the aqueous test solution, and the sertraline lactate solubility in the test solution are listed in Table 3-1 below. Solubility of sertraline lactate in water is approximately 125 mg/ml. The data below show that eight organic acids effected sertraline lactate solubilities about the same as or higher than 125 mg/ml; adipic, erythorbic, itaconic, citric, aspartic, glutamic, histidine, and ascorbic. Also, a solution of a mixture of two of these acids also had high solubility; ascorbic and aspartic. Sertraline lactate solubility was also high in calcium salt solutions, either alone (calcium citrate) or mixed with ascorbic acid.

TABLE 3-1

| | Excipient Concentration | Sertraline Lactate |
|---|---|---|
| Excipient | (mg/ml) | Solubility (mg/ml) |
| Adipic acid | 14 | 360 |
| Erythorbic acid | 400 | >217 |
| Itaconic acid | 150 | >202 |
| Citric acid | 600 | 162 |
| Aspartic acid | 7 | >155 |
| Glutamic acid | 12 | >125 |
| Histidine | 42 | >116 |
| Ascorbic/Aspartic | 400/7 | 116 |
| Ascorbic | 400 | 102 |
| Glycine | 250 | 66 |
| Aconitic acid | 200 | <59 |
| Tartaric acid | 1400 | 12 |
| Fumaric acid | 11 | <9 |
| Sorbic acid | 3 | <9 |
| Calcium lactate/ | 50/400 | 160 |
| Ascorbic acid | | |
| Calcium citrate | 10 | 165 |
| Calcium carbonate/ | 50/400 | 176 |
| Ascorbic acid | | |
| None | — | 125 |

### EXAMPLE 4

The lower solubility of the sertraline chloride salt and of all sertraline lactate and sertraline acetate salts in the presence of high chloride concentrations suggest that core formulations are preferred for which sertraline stays in solution that is, it does not precipitate or form a gel-like material when chloride is present. Certain organic acids and salts were found to inhibit precipitation or gelation of sertaline when chloride is present via the following screening test. Sertraline lactate was dissolved in water either alone (as a control) or with a candidate solubilizing agent. Sodium chloride was then added (as a concentrated solution) and the result observed. An excipient was considered beneficial if the solution remained clear and fluid. The more chloride that could be added to an excipient solution with the solution remaining clear, the more beneficial was the excipient. Table 4-1 below shows the results of this screening test, indicating that all the excipients tested increased sertraline concentration in the chloride solutions.

TABLE 4-1

| | | Concen- | Final | |
|---|---|---|---|---|
| | Excipient | tration | Sertraline | Observation |
| | Concentration | NaCl | Concentration | After NaCl |
| Excipient | (mg/ml) | (mM) | (mg/ml) | Addition |
| None | — | 38 | 22 | gel/ |
| | | | | precipitate |
| Ascorbic/ | 400/7 | 152 | 162 | solution |
| Aspartic acids | | | | |
| Aspartic acid | 7 | 114 | 162 | solution |
| | 7 | 152 | 100 | gel |
| Ascorbic acid | 400 | 100 | 102 | precipitate |
| Ascorbic acid/ | 400/50 | 150 | 165 | solution |
| calcium lactate | | | | |
| Ascorbic acid/ | 400/50 | 150 | 170 | slightly |
| calcium | | | | turbid |
| carbonate | | | | |
| Citric acid/ | 600/50 | 150 | 162 | solution |
| calcium lactate | | | | |
| Histidine | 42 | 150 | 110 | slight |
| | | | | precipitate |

### EXAMPLE 5

Organic compounds (solubilizers) were screened for their ability to enhance the solubility of sertraline lactate in aqueous solutions with or without the presence of chloride. Excess sertraline lactate was added to an aqueous solution of the candidate solubilizer and, in most cases an organic acid. The organic acids were saturated in these solutions and the additional solubilizing agents were at the concentration shown in Table 5-1. The equilibrium sertraline solubility was measured. Then, sodium chloride was added to the saturated solution and the final sertraline concentration was measured. The results of these screening tests are summarized in Table 5-1.

TABLE 5-1

| | | | | | | Sertraline |
|---|---|---|---|---|---|---|
| | | Solubilizer | | Sertraline | NaCl | Concentration |
| | | Concentration | Organic | Solubility | Concentration | with NaCl |
| | Solubilizer | (mg/ml) | Acid | (mg/ml) | (mM) | (mg/ml) |
| 1 | None (control) | — | none | 125 | 150 | 5 |
| 2 | Monocaprylin | 10 | ascorbic | 160 | 150 | 160 |
| 3 | Triacetin | 100 | ascorbic | 170 | 150 | 170 |
| 4 | Monobutyrin | 50 | none | 120 | 150 | 120 |
| 5 | Diacetin | 50 | ascorbic | 120 | 150 | 120 |
| 6 | Imwitor ® 312 | 10 | ascorbic | 120 | 150 | 120 |
| 7 | Imwitor ® 375 | 10 | ascorbic | 120 | 150 | 120 |
| 8 | Imwitor ® 742 | 50 | none | 120 | 150 | 120 |
| 9 | Imwitor ® 988 | 50 | none | 140 | 100 | 140 |
| 10 | Triethyl citrate | 50 | ascorbic | 160 | 150 | 160 |
| 11 | Pluronic ® L31 | 50 | none | 120 | 100 | 120 |
| 12 | Cremophore ® EL | 50 | ascorbic | 120 | 150 | 120 |
| 13 | Sucrose acetate | 50 | ascorbic | 120* | 150 | 120 |
| | isobutyrate | | | | | |
| 14 | Sodium capryl lactate | 50 | ascorbic | 120 | 150 | 120 |
| 15 | Sucrose monolaurate | 50 | none | 150 | 150 | 150 |
| 16 | Sodium lauryl lactate | 50 | ascorbic | 120 | 150 | 120 |
| 17 | Span 80 | 50 | ascorbic | 120 | 150 | 120 |

### EXAMPLE 6

This example illustrates that solubilizers for sertraline also can increase the rate of dissolution of sertraline. The effect of a candidate excipient on sertraline dissolution rate was determined by adding solid drug, the candidate solubilizing excipient, and, in some cases, other excipients such as an organic acid and an osmagent (such as a sugar) to a 1.8 ml centrifuge tube. The sample tubes were spun at 14K G for 5 minutes in a microcentrifuge to pack the powder. 150 μl gastric buffer was added to the packed powder and the samples were gently agitated, then spun at 14K G in a microcentrifuge for 2 minutes. The samples were then removed from the microcentrifuge and allowed to stand undisturbed until the solution was removed. The solution was removed from the samples after a total of 10 minutes after gastric buffer was added to the powder pack, and analyzed by HPLC to determine the sertraline concentration.

The dissolution rate (mg sertraline/ml-min) was calculated from the measured concentration of dissolved sertraline in the supernatant as a function of time over the first 10 minutes of dissolution. These dissolution rates and the excipient mixtures for which they were measured are summarized in Table 6-1 below. As shown, several excipient mixtures containing solubilizers significantly (about 3× or greater) increased the dissolution rate of sertraline, compared with sertraline alone and compared with sertraline and ascorbic acid.

TABLE 6-1

| Candidate Excipient | | | | | | | | | |
|---|---|---|---|---|---|---|---|---|---|
| | Concen- | | Organic | | | | Other | Sertraline | Sertraline |
| | tration | Organic | Acid Conc. | | Osmagent | Other | Excipient | Salt Form | Dissolution |
| Name | (wt %) | Acid | (wt %) | Osmagent | Conc. (wt %) | Excipient | Conc. (wt %) | Conc. (wt %) | Rate (mg/ml-min) |
| None | — | none | — | none | — | none | — | lactate | |
| | | | | | | | | 100 | |
| None | — | ascorbic | 51.0 | lactose | 20 | none | — | lactate | 3.5 |
| | | | | | | | | 14 | |
| Imwitor ® 312 | 5.0 | ascorbic | 49.5 | lactose | 12.5 | CaCO₃ | 5 | lactate | 20.9 |
| | | | | | | | | 14 | |
| Lecithin | 5.0 | ascorbic | 51.0 | lactose | 15 | none | — | lactate | 10 |
| | | | | | | | | 14 | |
| PEG 3550 | 5.0 | ascorbic | 51.0 | lactose | 15 | none | — | lactate | 9.3 |
| | | | | | | | | 14 | |
| Capmul ® MCM | 5.0 | ascorbic | 71.0 | none | — | none | — | lactate | 14.5 |
| | | | | | | | | 24 | |
| Capmul ® MCM | 4.7 | none | none | lactose | 17 | CaCO₃ | 4.7 | lactate | 4.3 |
| | | | | | | Ca citrate | 47 | 13.1 | |
| Imwitor ® 191 | 5.0 | ascorbic | 49.5 | lactose | 12.5 | CaCO₃ | 1.0 | lactate | 8.0 |
| | | | | | | | | 14 | |
| Myvrerol ® (18-99) | 5.0 | ascorbic | 49.5 | lactose | 12.5 | none | — | lactate | 6.4 |
| | | | | | | | | 14 | |
| Span ® 60 | 5.0 | ascorbic | 51.0 | lactose | 15 | none | — | lactate | 9.5 |
| | | | | | | | | 14 | |
| Ascorbyl | 6.8 | none | none | lactose | 74.2 | none | — | lactate | 4.3 |
| palmitate | | | | | | | | 19 | |
| Methyl paraben/ | 0.5/0.5/1.0 | ascorbic | 50.0 | lactose | 17.5 | none | — | lactate | 11.5 |
| propyl paraben/ | | | | | | | | 14 | |
| propyl gallate | | | | | | | | | |
| Imwitor ® 312 | 6.8 | aspartic | 7402 | none | — | none | — | lactate | 5.3 |
| | | | | | | | | 19 | |

### EXAMPLE 7

This examples illustrates a method for making osmotic tablets comprising a tablet core containing sertraline with and without solubilizing agents surrounded by a semipermeable asymmetric membrane coating. In this example the benefit of incorporating solubilizers into a controlled-release formulation containing sertraline is demonstrated. Sertraline-hydrochloride was triturated by hand for 10 minutes with citric acid and microcrystalline cellulose (Avicel PH 102, FMC) using a 6½ inch diameter mortar and pestle. Magnesium stearate was then blended in as a lubricant by stirring with a spatula for 60 seconds. The weight ratio of sertraline-hydrochloride to citric acid to microcrystalline cellulose to magnesium stearate was 8.5:63.8:23.7:4; with a total weight of 10 grams. The blended mixture was pressed into 470 mg tablets in a modified hydraulic jack (manufactured by Dayton) fitted with a pressure gauge and ⅜ inch concave punch under 2500 PSI pressure for 2 seconds. The dimensions of the resulting tablets were ⅜ inch in diameter and ¼ inch thick. A semipermeable membrane coating (as described in U.S. Pat. No. 5,612,059 was applied to these tablets using a LDCS-20 pan coater (Vector Corp.) at a spray rate of 20 grams per minute, an inlet temperature of 40° C. and air flow of 40 cfm. The coating solution contained by weight 10% Cellulose acetate, (Eastman Chemical, CA398-10), 2.5% polyethylene glycol (BASF, PEG 3350), 15% water and 72.5% acetone. The coated tablets were dried 1 hour at 50° C. before testing. After drying, the weight of applied coating material was 15.4% of the total weight. Additional osmotic delivery tablets were prepared by using essentially the same procedure for making the tablet cores and applying the asymmetric membrane coating to the cores described above. The composition of the cores and coating solution varied as shown in Table 7-1. Significant core compositional changes shown include: the sertraline salt form, the type and amount of solubilizer, and the type and amount of osmagent. The amount of binder (Avicel®) lubricant (magnesium stearate), and solubilizer were varied as necessary to obtain good tableting and wetting properties. These tablets all contained a sertraline dose of 50 mgA/tablet.

TABLE 7-1

| | Core Composition | | | | | | | | | | | | Coating Solution | | | | |
|---|---|---|---|---|---|---|---|---|---|---|---|---|---|---|---|---|---|
| Ex- | | | | Solubilizer | | | | | | | | | | | | | Coating |
| am- | Core | Drug | | Acid | | | | | | | Mg | | | | | | Weight |
| ple | Weight | Salt | Wt | | Wt | Solubilizer | | Osmogent | | Avicel | St. | | Polymer | Polymer | PEG | Water | (dry wt |
| No. | (mg) | Form | % | Type | % | Type | Wt % | Type | Wt % | wt % | wt % | Other | Type | wt % | wt % | wt % | %) |
| 7a | 470 | chloride | 12 | none | | none | | lactose | 66 | 20 | 2 | none | CA | 10 | 2.5 | 15 | 15.4 |
| 7b | 470 | lactate | 14 | none | | none | | lactose | 65.4 | 19.3 | 1.33 | none | EC | 6 | 4 | 8 | 1 |
| 7c | 470 | lactate | 14 | aspartic | 11 | none | | fructose | 38 | 29.5 | 2.5 | Ca | CA | 10 | 2.5 | 15 | 11 |
| | | | | | | | | | | | | Acetate | | | | | |
| 7d | 470 | lactate | 14 | glutamic | 10 | MC | 5 | sucrose | 50 | 15 | none | Ca | EC | 6 | 4 | 10 | 10.1 |
| | | | | | | | | | | | | lactate, | | | | | |
| | | | | | | | | | | | | Myrj | | | | | |
| 7e | 470 | lactate | 14 | aspartic | 11 | Im | 5 | fructose | 36 | 27 | 2.5 | Ca | CA | 10 | 2.5 | 15 | 10.3 |
| | | | | | | | | | | | | acetate | | | | | |
| 7f | 470 | lactate | 14 | glycine | 25 | Im | 5 | fructose | 28.5 | 25 | 2.5 | none | CA | 10 | 2.5 | 15 | 15.9 |
| 7g | 470 | lactate | 14 | aspartic | 11 | Im | 5 | fructose | 36 | 27 | 2.5 | Ca | CA | 10 | 2.5 | 15 | 20 |
| | | | | | | | | | | | | acetate | | | | | |
| 7h | 470 | lactate | 14 | aspartic | 11 | none | | fructose | 38 | 29.5 | 2.5 | Ca | CA | 10 | 2.5 | 15 | 10 |
| | | | | | | | | | | | | acetate | | | | | |

IM = Imwitor ® 312
CA = cellulose acetate 398-10
EC = Ethocel S-100
MC = monocaprylin
Mg St. = magnesium stearate
PEG = polyethylene glycol 3350
Myrj = Myrj ® 52

The rates of release of sertraline from these formulations were determined testing the tablets in a USP Apparatus #2 with paddle stirring speed set at 100 rpm. The receptor solution used in the dissolution apparatus was 0.13M acetate buffer at pH 4.0 with 0.075M sodium chloride kept at 37° C. Samples of the receptor solution were taken at the times shown in Table 7-2. Analysis of sertraline released was determined by reverse-phase high-performance liquid chromatography (RP HPLC).

The results of release-rate tests performed using these procedures are listed in Table 7-2. The first two formulations listed, 7a and 7b show low release rates and are included as comparison examples. Both these formulations contain a sertraline salt (hydrochloride or lactate) and only lactose as the osmagent and no solubilizing excipients. The remaining formulations (7c-7h) listed in Table 7-2 all contain one or more solubilizing excipients and all demonstrate significantly higher release rates of sertraline compared with the formulations that do not contain solubilizers.

TABLE 7-2

| Tablets of | Fraction of Drug Released (%) At Specified Time | | | | | | |
|---|---|---|---|---|---|---|---|
| Example No | 0 Hr | 1 Hr | 2 Hr | 4 Hr | 8 Hr | 12 Hr | 20 Hr |
| 7a | 0 | 0 | 0 | 0 | 0 | 0 | 0 |
| 7b | 0 | 0 | 1 | 2 | — | 10 | 12 |
| | | | | | | (17 hr) | |
| 7c | 0 | 6 | 15 | 35 | 62 | 76 | 78 |
| 7d | 0 | 0 | 0 | 4 | 19 | 28 | 44 |
| 7e | 0 | 8 | 19 | 37 | 60 | 73 | 83 |
| 7f | 0 | 0.7 | 6 | 17 | 37 | 54 | 78 |
| 7g | 0 | 0.4 | 4 | 13 | 31 | 41 | 53 |
| 7h | 0 | 8 | 18 | 38 | 56 | 64 | 66 |

1. A composition of matter comprising a highly soluble salt form of sertraline having a solubility in pure water of greater than 10 mgA/ml, and an amount of a solubilizing agent sufficient to produce a concentration of dissolved sertraline in a use environment containing chloride ions which is 1.5 times higher than the concentration effected by a comparative composition of matter identical thereto but for the inclusion of said solubilizing agent.

2. (original)

3. (original)

4. (original)

5. (original)

6. (original)

7. A composition of matter as defined in claim 1, wherein said solubilizing agent is selected from the group consisting of:
1) organic acids and organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers
9) sorbitan esters;
10) polyoxyethylene sorbitan esters; and
11) calcium salts.

8. (original)

9. A composition as defined in claim 1, wherein said solubilizing agent is selected from aspartic acid, glyceryl monocaprylate, glyceryl monolaurate, calcium acetate, ascorbic acid, citric acid, and glutamic acid.

10. A composition of matter comprising a highly soluble salt form of sertraline having a solubility in pure water of greater than 10 mgA/ml, and an amount of a solubilizing agent sufficient to maintain, for at least 2 hours in 0.075M sodium chloride, a concentration of dissolved sertraline in a use environment containing chloride ions which is 1.5 times higher than the concentration effected by a comparative composition of matter identical thereto but for the inclusion of said solubilizing agent.

11. (original)

12. (original)

13. A composition of matter as defined in claim 10, wherein said solubilizing agent is selected from the group consisting of:
1) organic acids and organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers
9) sorbitan esters;
10) polyoxyethylene sorbitan esters; and
11) calcium salts.

14. A composition as defined in claim 1, wherein said solubilizing agent is selected from aspartic acid, glyceryl monocaprylate, glyceryl monolaurate, calcium acetate, ascorbic acid, citric acid, and glutamic acid.

15. A composition of matter comprising a highly soluble salt form of sertraline having a solubility in pure water of greater than 10 mgA/ml, and an amount of a solubilizing agent sufficient to effect, in vivo, a Cₘₐₓ and/or an AUC which is greater by at least 10% than the Cₘₐₓ and/or AUC effected by a comparative composition of matter identical thereto but for the inclusion of said solubilizing agent.

16. (original)

17. (original)

18. (original)

19. (original)

20. A composition of matter as defined in claim 15, wherein said solubilizing agent is selected from the group consisting of:
1) organic acids and organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers
9) sorbitan esters;
10) polyoxyethylene sorbitan esters; and
11) calcium salts.

21. A composition of matter as defined in claim 15, wherein said solubilizing agent is selected from aspartic acid, glyceryl monocaprylate, glyceryl monolaurate, calcium acetate, ascorbic acid, citric acid, and glutamic acid.

22. A method of increasing the solubility of sertraline in an aqueous chloride ion-containing use environment, comprising administering said sertraline to said use environment in a composition of matter additionally comprising a solubilizing agent, wherein said sertraline is in the form of a highly soluble salt form having a solubility in pure water of greater than 10 mgA/ml.

23. (original)

24. (original)

25. (original)

26. (original)

27. (original)

28. (original)

29. A composition of matter as defined in claim 22, wherein said solubilizing agent is selected from the group consisting of:
1) organic acids and organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers
9) sorbitan esters;
10) polyoxyethylene sorbitan esters; and
11) calcium salts.

30. The composition of claim 1 wherein said highly soluble salt form of sertraline is selected from the group consisting of sertraline aspartate, sertraline acetate, and sertraline lactate.

31. The composition of claim 10 wherein said highly soluble salt form of sertraline is selected from the group consisting of sertraline aspartate, sertraline acetate, and sertraline lactate.

32. The composition of claim 15 wherein said highly soluble salt form of sertraline is selected from the group consisting of sertraline aspartate, sertraline acetate, and sertraline lactate.

33. The composition of claim 1 wherein said solubilizing agent is selected from the group consisting of adipic acid, erythorbic acid, itaconic acid, citric acid, ascorbic acid, aspartic acid, glutamic acid, and lactic acid.

34. The composition of claim 10 wherein said solubilizing agent is selected from the group consisting of adipic acid, erythorbic acid, itaconic acid, citric acid, ascorbic acid, aspartic acid, glutamic acid, and lactic acid.

35. The composition of claim 15 wherein said solubilizing agent is selected from the group consisting of adipic acid, erythorbic acid, itaconic acid, citric acid, ascorbic acid, aspartic acid, glutamic acid, and lactic acid.

36. The composition of claim 33 further comprising another solubilizer selected from the group consisting of 1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers;
9) sorbitan esters;
10) polyoxyethylene sorbitan esters; and
11) calcium salts.

37. The composition of claim 34 further comprising another solubilizer selected from the group consisting of
1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers;
9) sorbitan esters;
10) polyoxyethylene sorbitan esters; and
11) calcium salts.

38. The composition of claim 35 further comprising another solubilizer selected from the group consisting of
1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers;
9) sorbitan esters;
10) polyoxyethylene sorbitan esters; and
11) calcium salts.

39. A composition of matter comprising sertraline or a pharmaceutically acceptable salt form of sertraline and an amount of a solubilizing agent sufficient to produce a concentration of dissolved sertraline in a use environment containing chloride ions which is 1.5 times higher than the concentration effected by a comparative composition of matter identical thereto but for the inclusion of said solubilizing agent, wherein said solubilizing agent has a solubility of at least 1 mg/ml in said use environment and wherein said solubilizing agent is an organic acid.

40. A composition of matter as defined in claim 39, wherein said use environment is the GI tract.

41. A composition of matter as defined in claim 39, wherein said use environment is an aqueous chloride ion-containing test medium.

42. A composition of matter as defined in claim 41, wherein said use environment is 0.075 M sodium chloride.

43. A composition of matter as defined in claim 39, which is an immediate release dosage form.

44. A composition of matter as defined in claim 39, which is a controlled release dosage form.

45. A composition of matter as defined in claim 39, further comprising another solubilizing agent selected from:
1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers
9) sorbitan esters;
10) polyoxyethylene sorbitan esters; and
11) calcium salts.

46. A composition of matter as defined in claim 42, wherein the amount of said solubilizing agent is sufficient to maintain, for at least 2 hours, the concentration of dissolved sertraline at a level which is at least 1.5 times higher than the concentration of sertraline produced by a comparative composition of matter identical thereto but for the inclusion of said solubilizing agent.

47. A composition as defined in claim 39, wherein said solubilizing agent is selected from malic acid, citric acid, erythorbic acid, adipic acid, maleic acid, aspartic acid, tartaric, and glutamic acid.

48. A composition of matter comprising sertraline or a pharmaceutically acceptable salt thereof and an amount of a solubilizing agent sufficient to produce and to maintain, for at least 2 hours in 0.075M sodium chloride, a concentration of dissolved sertraline which is at least 1.5 times higher than the concentration effected by a comparative composition of matter identical thereto but for the inclusion of said solubilizing agent, wherein said solubilizing agent has a solubility of at least 1 mg/ml in said use environment and wherein said solubilizing agent is an organic acid.

49. A composition of matter as defined in claim 48, which is an immediate release dosage form.

50. A composition of matter as defined in claim 48, which is a controlled release dosage form.

51. A composition of matter as defined in claim 48, further comprising another solubilizing agent selected from the group consisting of:
1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers;
9) sorbitan esters;
10) polyoxyethylene sorbitan esters; and
11) calcium salts.

52. A composition as defined in claim 48, wherein said solubilizing agent is selected from the group consisting of malic acid, citric acid, erythorbic acid, adipic acid, maleic acid, aspartic acid, tartaric, and glutamic acid.

53. A composition of matter comprising sertraline or a pharmaceutically acceptable salt thereof and an amount of a solubilizing agent sufficient to effect, in vivo, a Cₘₐₓ and/or an AUC which is greater by at least 10% than the Cₘₐₓ and/or AUC effected by a comparative composition of matter identical thereto but for the inclusion of said solubilizing agent, wherein said solubilizing agent has a solubility of at least 1 mg/ml in said use environment and wherein said solubilizing agent is an organic acid.

54. A composition as defined in claim 53, wherein said Cₘₐₓ and/or AUC effected by said solubilizing agent-containing composition is at least 15% higher than the corresponding Cₘₐₓ and/or AUC effected by said comparative composition.

55. A composition as defined in claim 53, wherein said Cₘₐₓ and/or AUC effected by said solubilizing agent-containing composition is at least 20% higher than the corresponding Cₘₐₓ and/or AUC effected by said comparative composition.

56. A composition of matter as defined in claim 53, which is an immediate release dosage form.

57. A composition of matter as defined in claim 53, which is a controlled release dosage form.

58. A composition of matter as defined in claim 53, further comprising another solubilizing agent selected from the group consisting of:
1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers;
9) sorbitan esters;
10) polyoxyethylene sorbitan esters; and
11) calcium salts.

59. A composition of matter as defined in claim 53, wherein said solubilizing, agent is selected from the group consisting of malic acid, citric acid, erythorbic acid, adipic acid, maleic acid, aspartic acid, tartaric, and glutamic acid.

60. A method of increasing the solubility of sertraline in an aqueous chloride ion-containing use environment, comprising administering said sertraline to said use environment in a composition of matter additionally comprising a solubilizing agent, wherein said solubilizing agent has a solubility of at least 1 mg/ml in said use environment and wherein said solubilizing agent is an organic acid.

61. A method as defined in claim 60, wherein the concentration of dissolved sertraline in said use environment also containing said solubilizer is at least 1.5-fold higher than the concentration of sertraline effected by a comparative composition identical to said solubilizing agent-containing composition except for the inclusion of said solubilizing agent.

62. A method as defined in claim 60, wherein said use environment is the GI tract.

63. A method as defined in claim 60, wherein said use environment is an aqueous chloride ion-containing test medium.

64. A method as defined in claim 63, wherein said medium is 0.075M sodium chloride.

65. A method as defined in claim 60, wherein said composition of matter is in the form of an immediate release dosage form.

66. A method as defined in claim 60, wherein said composition of matter is in the form of a controlled release dosage form.

67. A method as defined in claim 60, further comprising another solubilizing agent selected from the group consisting of:
1) organic acid salts;
2) partial glycerides;
3) glycerides;
4) glyceride derivatives;
5) polyethylene glycol esters;
6) polypropylene glycol esters;
7) polyhydric alcohol esters;
8) polyoxyethylene ethers;
9) sorbitan esters;
10) polyoxyethylene sorbitan esters; and
11) calcium salts.
